(19) United States
(12) Patent Application Publication
(10) Pub. No.: US 2012/0272962 A1
(43) Pub. Date: Nov. 1, 2012

(54) METHODS AND SYSTEMS FOR MANAGING A VENTILATOR PATIENT WITH A CAPNOMETER

(75) Inventors: Peter Doyle, Vista, CA (US); Joseph Doug Vandine, Manteca, CA (US); Warren Sanborn, Escondido, CA (US); Dan Graboi, Encinitas, CA (US)

(73) Assignee: Nellcor Puritan Bennett LLC, Boulder, CO (US)

(21) Appl. No.: 13/098,152

(22) Filed: Apr. 29, 2011

(51) Int. Cl. A61M 16/00

(52) U.S. Cl. 128/204.23

(57) ABSTRACT

This disclosure describes systems and methods for managing the ventilation of a patient being ventilated by a medical ventilator. The disclosure describes a novel approach of displaying integrated ventilator information with capnometer data. The disclosure further describes a novel approach for determining if the ventilator breathing circuit is occluded or disconnected.

### INTRODUCTION

Medical ventilator systems have long been used to provide supplemental oxygen support to patients. These ventilators typically comprise a source of pressurized oxygen which is fluidly connected to the patient through a conduit. Some ventilator systems monitor the patient during ventilation. In some systems, carbon dioxide (CO₂) levels in the breathing gas from the patient are measured.

Many of these previously known medical ventilators display the monitored CO₂ levels of the breathing gas from the patient. While these previously known ventilation systems display CO₂ readings or capnometer data, patient care could be improved by further coordinating the operation of the two devices, particularly by integrating the analysis, storage and display of particular aspects of carbon dioxide data and respiratory data.

### SUMMARY

This disclosure describes systems and methods for managing the ventilation of a patient being ventilated by a medical ventilator. The disclosure describes a novel approach of displaying integrated ventilator information with capnometer data. The disclosure further describes a novel approach for determining if the ventilator breathing circuit is occluded or disconnected.

In part, this disclosure describes a method for managing ventilation of a patient being ventilated by a medical ventilator. The method including:

a) monitoring at least one CO₂ parameter;

b) monitoring breathing circuit pressure;

c) monitoring exhaled flow and calculating exhaled volume therefrom;

d) determining that the at least one CO₂ parameter is less than a predetermined CO₂ threshold amount, the exhaled pressure is less than a predetermined threshold pressure, and the exhaled volume is less than a predetermined threshold volume; and

e) executing a disconnection alarm.

The disclosure also describes another method for managing ventilation of a patient being ventilated by a medical ventilator. The method includes:

a) monitoring at least one CO₂ parameter of gas in the patient circuit;

b) monitoring at least one of exhaled volume and delivered volume;

c) determining that the at least one CO₂ parameter drops by a predetermined amount in a predetermined amount of time concurrently with a drop in the at least one of the exhaled volume by a predetermined amount and the delivered volume by a predetermined amount; and

d) executing an occlusion alarm

Yet another aspect of this disclosure describes a medical ventilator-capnometer system including:

a) a pneumatic gas delivery system, the pneumatic gas delivery system adapted to control a flow of gas from a gas supply to a patient via a ventilator breathing circuit;

b) a flow sensor;

c) a pressure sensor;

d) a capnometer, the capnometer monitors an amount of carbon dioxide in the respiration gas from the patient in the ventilator breathing circuit in order to monitor VCO₂ and ETCO₂;

e) a breathing circuit module, the breathing circuit module determines that concurrently at least one of the VCO₂ and the ETCO₂ are below a predetermined amount, pressure is below a predetermined amount, and an exhaled volume is below a predetermined amount in the ventilator breathing circuit based on flow sensor readings, pressure sensor readings, and capnometer readings before executing a disconnection alarm; and

a processor in communication with the pneumatic gas delivery system, the flow sensor, the pressure sensor, the capnometer, and the breathing circuit module.

The disclosure also describes a medical ventilator-capnometer system that includes:

a) a pneumatic gas delivery system, the pneumatic gas delivery system adapted to control a flow of gas from a gas supply to a patient via a ventilator breathing circuit;

b) a flow sensor;

c) a capnometer, the capnometer monitors an amount of carbon dioxide in the respiration gas from the patient in the ventilator breathing circuit in order to monitor VCO₂ and ETCO₂;

d) a breathing circuit module, the breathing circuit module determines that at least one of the VCO₂ and the ETCO₂ drops by a predetermined amount within a predetermined amount of time, concurrently as at least one of delivered volume and exhaled volume drop by a predetermined amount in the ventilator breathing circuit based on flow sensor readings and capnometer readings before executing an occlusion alarm; and

e) a processor in communication with the pneumatic gas delivery system, the flow sensor, the capnometer, and the breathing circuit module.

The disclosure further describes a computer-readable medium having computer-executable instructions for performing a method for managing ventilation of a patient being ventilated by a medical ventilator-capnometer system. The method includes:

a) repeatedly monitoring at least one CO₂ parameter, the at least one CO₂ parameter comprises ETCO₂ and VCO₂;

b) repeatedly monitoring breathing circuit pressure;

c) repeatedly monitoring exhaled volume;

d) repeatedly determining that the at least one CO₂ parameter is less than a predetermined threshold amount, the exhaled pressure is less than a predetermined pressure threshold, and the exhaled volume is less than a predetermined volume threshold; and

e) repeatedly executing a disconnection alarm.

In yet another aspect, the disclosure describes a medical ventilator-capnometer system that includes:

a) means for monitoring at least one CO₂ parameter, the at least one CO₂ parameter comprises ETCO₂ and VCO₂;

b) means for monitoring at least one of exhaled volume and delivered volume;

c) means for determining that the at least one CO₂ parameter drops by a predetermined amount in a predetermined amount of time concurrently with a drop in the at least one of the exhaled volume by a predetermined amount and the delivered volume by a predetermined amount; and

d) means for executing an occlusion alarm.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments, systems, and methods described below and are not meant to limit the scope of the invention in any manner, which scope shall be based on the claims appended hereto.

FIG. 1 illustrates an embodiment of a ventilator-capnometer system connected to a human patient.

FIG. 2 illustrates an embodiment of a method for managing the ventilation of a patient being ventilated by a medical ventilator-capnometer system.

FIG. 3 illustrates an embodiment of a method for managing the ventilation of a patient being ventilated by a medical ventilator-capnometer system.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, it is desirable to monitor the CO₂ levels in the respiration gas from the patient. Accordingly, ventilator systems may have capnometers for non-invasively determining the concentrations and/or pressures of CO₂ in the respiration gases from a patient, such as end tidal CO₂ or the amount of carbon dioxide released during exhalation and at the end of expiration (ETCO₂).

As known in the art, capnometers are devices for measuring CO₂ in a gas stream. In one common design, the capnometer utilizes a beam of infra-red light, which is passed across the ventilator circuit and onto a sensor, to determine the level of CO₂ in a patient's respiration gasses. As the amount of CO₂ in the respiration gas increases, the amount of infra-red light that can pass through the respiration gas and onto the sensor decreases, which changes the voltage in a circuit. The sensor utilizes the change in voltage to calculate the amount of CO₂ contained in the gas. Other designs are known in the art and any capnometry technology, now known or later developed, may be used in the embodiments described herein to obtain CO₂ readings.

Although ventilators and capnometers have been previously utilized on the same patient, ventilators typically display data based solely on ventilator data monitored by the ventilator. Further, capnometers typically display data based solely on the CO₂ readings. However, it is desirable to provide information that incorporates capnometer data with ventilator data to the patient, ventilator operator, and/or medical caregiver.

The present disclosure describes ventilator-capnometer systems and methods for managing the ventilation of a patient. The ventilator-capnometer systems described herein integrate capnometric data with ventilator data to provide the operator, medical care giver, and/or the patient with more precise patient information for the treatment and ventilation of the patient.

An embodiment of the ventilator-capnometer systems described herein is a system that is capable of managing the ventilation of a patient by monitoring ETCO₂, net volume of CO₂ exhaled by the patent (VCO₂), exhalation pressure, and/or exhaled volume to determine if the patient breathing circuit has been disconnected from the patient. In an additional embodiment of the ventilator-capnometer systems described herein, is a system that is capable of managing the ventilation of a patient by monitoring ETCO₂ or VCO₂ and exhaled volume and/or delivered volume to determine if the ventilator circuit or patient interface is occluded.

As observed in several clinical cases, the breathing circuit may become disconnected during patient ventilation. Previously utilized systems often rely on pressure and flow sensor readings to determine if a patient circuit has become disconnected or occluded. However, there is often a delay between a patient circuit disconnect or occlusion and an alarm generated by the monitoring of pressure and flow in the patient circuit. Further, the monitoring of pressure and flow in the patient circuit can also on occasion set off the disconnect alarm or occlusion alarm when the breathing circuit is not occluded and/or still attached or in other words can generate false alarms.

The monitoring of ETCO₂ and/or VCO₂ along with exhaled pressure and exhaled volume may be utilized to more quickly and more accurately determine a disconnection in a ventilator circuit than the monitoring of just pressure and flow in the breathing circuit to determine disconnection of the breathing circuit. Further, the monitoring of ETCO₂ and/or VCO₂ along with at least one of exhaled volume and delivered volume may be utilized to more quickly and more accurately determine an occluded ventilator circuit tubing or patient interface than the monitoring of just pressure and flow in the breathing circuit to determine occlusion of the breathing circuit or patient interface. The monitoring of these components also reduces the number of false alarms compared to the monitoring of just flow and pressure.

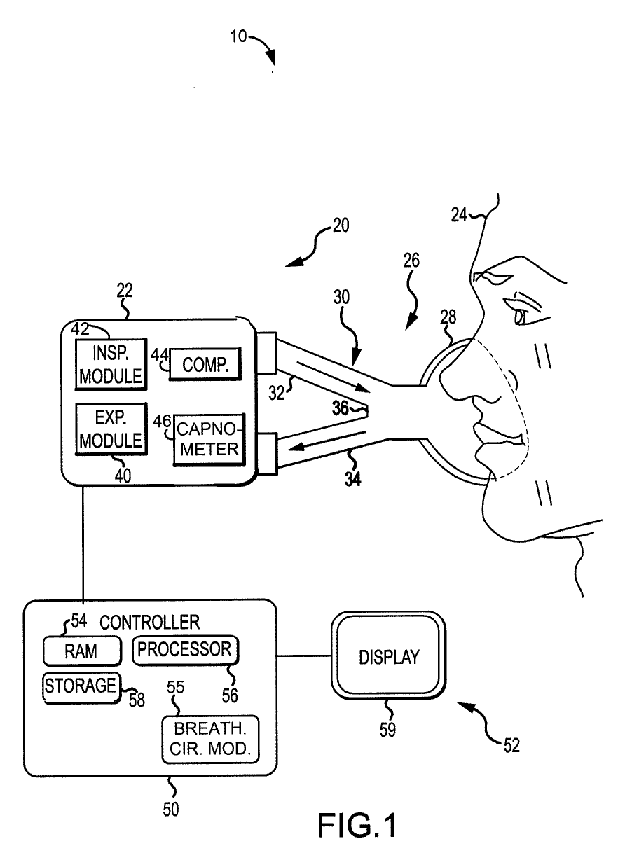

FIG. 1 illustrates an embodiment of a ventilator-capnometer system 10 attached to a human patient 24. The ventilator-capnometer system 10 includes a ventilator 20 in communication with a capnometer 46. As shown in FIG. 1 the capnometer 46 may be an integral part of ventilator 20. In an alternative embodiment, the capnometer 46 may be a separate component from ventilator 20.

Ventilator 20 includes a pneumatic gas delivery system 22 (also referred to as a pressure generating system 22) for circulating breathing gases to and from patient 24 via the ventilation tubing system 26, which couples the patient 24 to the pneumatic gas delivery system 22 via physical patient interface 28 and ventilator breathing circuit 30.

Ventilator breathing circuit 30 could be a two-limb or one-limb circuit 30 for carrying gas to and from the patient 24. In a two-limb embodiment as shown, a wye fitting 36 may be provided as shown to couple the patient interface 28 to the inspiratory limb 32 and the expiratory limb 34 of the ventilator breathing circuit 30. Examples of suitable patient interfaces 28 include a nasal mask, nasal/oral mask (which is shown in FIG. 1), nasal prong, full-face mask, tracheal tube, endotracheal tube, nasal pillow, etc.

Pneumatic gas delivery system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressured air and/or oxygen) is controlled through the use of one or more pneumatic gas delivery systems, such as a gas regulator.

Pneumatic gas delivery system 22 may include a variety of other components, including sources for pressurized air and/or oxygen, mixing modules, valves, sensors, tubing, filters, etc. In one embodiment, the pneumatic gas delivery system 22 includes at least one of a flow sensor and pressure sensor in the ventilator breathing circuit 30.

Capnometer 46 is in data communication with ventilator 20. This communication allows the ventilator 20 and capnometer 46 to send data, instructions, and/or commands to each other. Capnometer 46 is in communication with processor 56 of ventilator 20.

Capnometer 46 monitors the concentrations of carbon dioxide in the respiratory gas with a carbon dioxide sensor located in the ventilator breathing circuit 30. The carbon dioxide sensor allows the capnometer 46 to monitor in real-time the concentration of CO₂ in the gas transiting its sensor. Using this in conjunction with flow and/or volume signals, the system can calculate volumetric carbon dioxide (VCO₂), end-tidal carbon dioxide (ETCO₂), and minute volume. In one embodiment, capnometer 46 generates a capnogram with these data.

Controller 50 is in communication with pneumatic gas delivery system 22, capnometer 46, display 59, and an operator interface 52, which may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as non-transitory computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

In one embodiment, as illustrated in FIG. 1, the controller 50 further includes a breathing circuit module 55. In an alternative embodiment, not shown, the breathing circuit module 55 is a separate component from or independent of controller 50. In another embodiment, not shown, the breathing circuit module 55 is a separate component from or independent of ventilator 20.

The breathing circuit module 55 monitors sensor readings taken by the pressure sensor, the flow sensor, and the capnometer 46. The breathing circuit module 55 determines if VCO₂ and/or ETCO₂ are below a predetermined threshold concurrently with pressure at the same time that exhaled volume is also below a predetermined threshold. If breathing circuit module 55 determines that the VCO₂ and/or ETCO₂ are below the predetermined threshold concurrently with pressure and exhaled volume being below the predetermined threshold, breathing circuit module 55 executes a disconnection alarm. The disconnection alarm indicates that the ventilator breathing circuit 30 is disconnected. If breathing circuit module 55 determines that either the VCO₂ and/or ETCO₂ are not below the predetermined threshold or concurrently that the pressure and exhaled volume are not below their respective thresholds, breathing circuit module 55 continues to monitor sensor readings taken by the pressure sensor, the flow sensor, and the capnometer 46 and does not execute a disconnection alarm.

Additionally, the breathing circuit module 55 determines if VCO₂ and/or ETCO₂ drop by a predetermined amount in a predetermined amount of time concurrently with a drop in at least one of exhaled volume by a predetermined amount and delivered volume by a predetermined amount. If breathing circuit module 55 determines that the VCO₂ and/or ETCO₂ dropped by the predetermined amount in the predetermined amount of time concurrently with a drop in the least one of exhaled volume and delivered volume by their respective predetermined amounts, breathing circuit module 55 executes an occlusion alarm. The occlusion alarm indicates that the ventilator breathing circuit 30 is occluded. If breathing circuit module 55 determines that the VCO₂ and/or ETCO₂ did not drop by the predetermined amount in the predetermined amount of time or concurrently the at least one of exhaled volume and delivered volume did not drop by their predetermined amounts, breathing circuit module 55 continues to monitor sensor readings taken by the pressure sensor, the flow sensor, and the capnometer 46 and does not execute an occlusion alarm.

In one embodiment, the predetermined amounts, whether absolute thresholds or amounts of drop, are input by the operator. In another embodiment, the predetermined amounts are selected by the operator. In an alternative embodiment, the predetermined amounts are preconfigured and determined by the ventilator 20.

The alarm executed by the breathing circuit module 55 may be any suitable notification for gaining the attention of the medical care-giver, ventilator operation, and/or patient 24. In one embodiment, the alarm is any visual, audio, and/or vibrational notification. The alarm may be executed on the ventilator 20 or capnometer 46.

In the depicted example, operator interface 52 includes a display 59 that is touch-sensitive, enabling the display 59 to serve both as an input user interface and an output device. In an alternative embodiment, the display 59 is not touch sensitive or an input user interface. The display 59 can display any type of ventilation information, such as sensor readings, parameters, commands, alarms, warnings, and/or smart prompts (i.e., ventilator determined operator suggestions). Further, in one embodiment, display 59 displays an alarm executed by the breathing circuit module 55.

In an alternative embodiment, not shown, the capnometer 46 includes a display. In one embodiment, the capnometer display displays the alarm executed by the breathing circuit module 55.

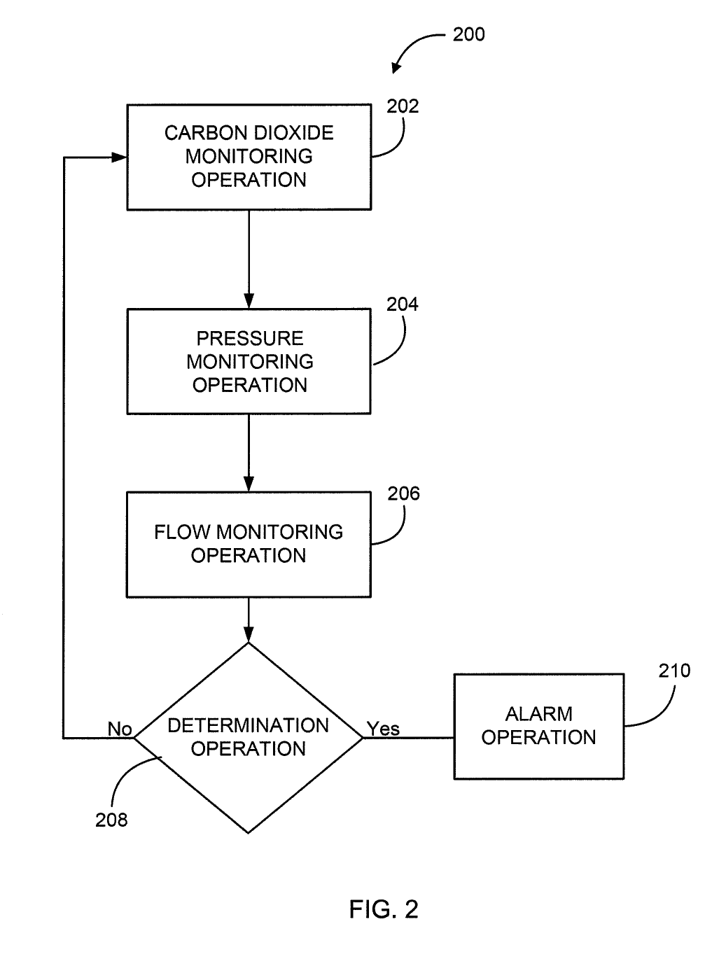

FIG. 2 illustrates an embodiment of a method 200 for managing a patient being ventilated by a medical ventilator-capnometer system. As illustrated, method 200 performs a carbon dioxide monitoring operation 202. Carbon dioxide monitoring operation 202 monitors the amount of carbon dioxide in the respiration gas of the ventilator patient. The capnometer utilizes a carbon dioxide sensor in the breathing circuit to monitor the amount of carbon dioxide in the respiration gas of the ventilator patient. The carbon dioxide sensor allows the capnometer to monitor in real-time at least one CO₂ parameter. In an embodiment, the CO₂ monitoring operation 202 includes taking a CO₂ measurement of the gas in the patient circuit periodically using a capnometer and from this data calculating a monitored CO₂ parameter such as VCO₂ and/or ETCO₂.

Further, method 200 performs a pressure monitoring operation 204. Pressure monitoring operation 204 monitors the pressure in the ventilator breathing circuit with one or more pressure sensors. The pressure may be monitored using a proximal pressure sensor or sensors near the patient wye or at any location or multiple locations in the patient circuit. Alternatively or in addition, the pressure may be monitored at the distal end of the exhalation limb and/or the inhalation limb.

Method 200 also performs a flow monitoring operation 206. Flow monitoring operation 206 monitors the flow of breathing gas delivered to and/or received from the patient in the breathing circuit with one or more flow sensors. The flow sensors allow the flow monitoring operation 206 to monitor in real-time exhaled volume and/or delivered volume. As with the CO₂ and pressure monitoring operations 202 and 204, the flow at any point or points in the patient circuit may be monitored. In an embodiment, the flow monitoring operation 202 includes integrating the flow data to calculate an exhaled volume. In an alternative embodiment, such a calculation may be performed separately as an independent operation or as part of the determination operation 208.

It should be noted that the monitoring operations 202, 204, 206 need not be performed in the order described above. Rather, the operations could be performed in any order including being performed simultaneously or as one, combined monitoring operation.

Method 200 also performs a determination operation 208. Determination operation 208 determines if the at least one CO₂ parameter is below a predetermined threshold amount concurrently with pressure and exhaled volume being below a predetermined threshold amount. If determination operation 208 determines that the at least one CO₂ parameter is below the predetermined threshold amount concurrently with pressure and exhaled volume being below their predetermined thresholds, the method 200 performs alarm operation 210. If the determination operation 208 determines that the at least one CO₂ parameter is not below the predetermined threshold amount or concurrently pressure and/or exhaled volume are not below their predetermined threshold amounts, the method 200 returns to the monitoring operations 202, 204, 206.

In performing the determination operation 208, the method 200 may perform multiple calculations. For example, pressure at a specific location may be calculated from measurements taken at other location(s) and all measurements may be modified to take into account temperature and humidity effects or to convert the measurements to a usable form or desired units.

Alarm operation 210 executes a disconnection alarm. The disconnection alarm signifies that the breathing circuit is disconnected from the ventilator-capnometer system. The disconnection alarm may be any suitable notification for gaining the attention of the medical caregiver, ventilator operator, and/or the patient. In one embodiment, the disconnection alarm is any suitable visual, audio, and/or vibrational notification.

Depending on how the method 200 is implemented, a ventilator could perform the method every computing cycle, once for every set number of cycles, or at specific points in the therapy, e.g., after every breath or specified phase of a breath (e.g. at the end of exhalation).

Thresholds should be selected so that false alarms are minimized. For example, a VCO2 threshold should be selected such that measured VCO2 dropping below the threshold means that it is highly unlikely a patient is breathing into the patient circuit. In one embodiment, method 200 receives the predetermined threshold amounts of ETCO₂, VCO₂, pressure, and/or exhaled volume from operator input. In an additional embodiment, the predetermined amounts are selected by the operator. In an alternative embodiment, the predetermined amounts are preconfigured and determined by the ventilator.

In one embodiment, method 200 performs a display operation. Display operation displays the disconnection alarm on a ventilator display. In another embodiment, display operation of method 200 displays the disconnection alarm on a capnometer display.

In one embodiment, method 200 is performed by the medical ventilator-capnometer system illustrated in FIG. 1 and described above.

In an alternative embodiment, a computer-readable medium having computer-executable instructions for performing methods for managing the ventilation of a patient being ventilated by a medical ventilator-capnometer system are disclosed. These methods include repeatedly performing the steps illustrated in FIG. 2 and as described in the description of FIG. 2 above.

In another embodiment, the medical ventilator-capnometer system includes: means for monitoring at least one CO₂ parameter, the at least one CO₂ parameter comprises ETCO₂ and VCO₂; means for monitoring exhaled pressure; means for monitoring exhaled volume; means for determining that at least one CO₂ parameter, the exhaled pressure, and the exhaled volume are all less than predetermined amounts; and means for executing a disconnection alarm. In one embodiment, the means for the medical ventilator-capnometer system are illustrated in FIG. 1 and described in the above description of FIG. 1. However, the means described above for FIG. 1 and illustrated in FIG. 1 are but one example only and are not meant to be limiting.

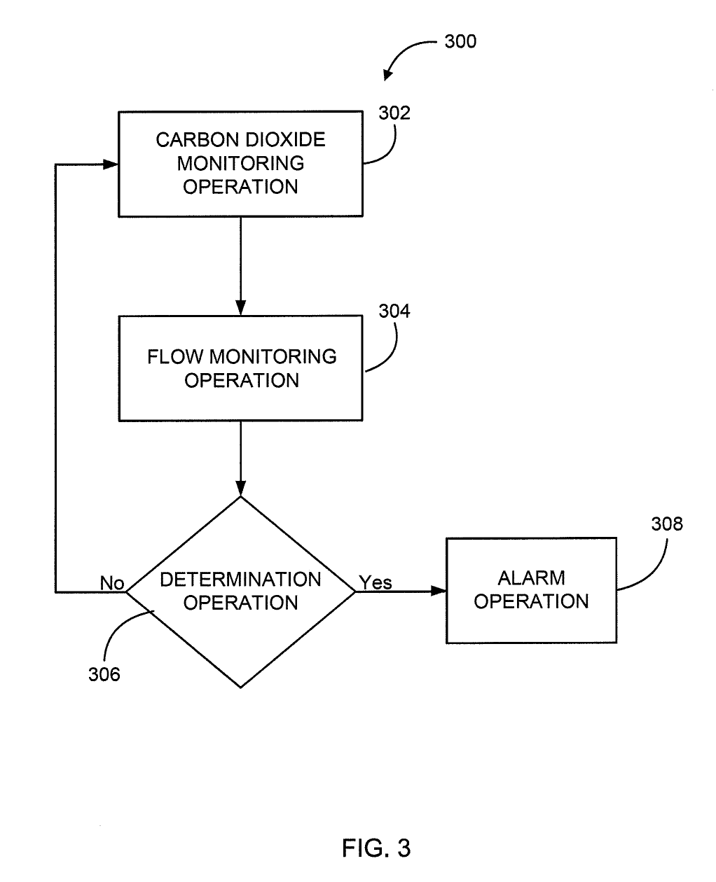

FIG. 3 illustrates another embodiment of a method 300 for managing a patient being ventilated by a medical ventilator-capnometer system. As illustrated, method 300 performs a carbon dioxide monitoring operation 302. Carbon dioxide monitoring operation 302 monitors the amount of carbon dioxide in the respiration gas of the ventilator patient. The carbon dioxide monitoring operation 302 is substantially as described above with reference to FIG. 2. The capnometer utilizes a carbon dioxide sensor in the breathing circuit to monitor the amount of carbon dioxide in the respiration gas of the ventilator patient. The carbon dioxide sensor allows the capnometer to monitor in real-time at least one CO₂ parameter. The at least one CO₂ parameter includes volumetric carbon dioxide (VCO₂) and/or end-tidal carbon dioxide (ETCO₂).

Further, method 300 performs a flow monitoring operation 304 substantially as described above with reference to FIG. 2. In an alternative embodiment, a pressure monitoring operation (not shown) may also be performed. Again, the monitoring operations 302, 304 need not be performed in the order described above. Rather, the operations could be performed in any order including being performed simultaneously or as one combined monitoring operation.

Method 300 also performs a determination operation 306. Determination operation 306 determines if the at least one CO₂ parameter drops by a predetermined amount in a predetermined amount of time concurrently with a predetermined drop in delivered volume and/or a predetermined drop in exhaled volume. If determination operation 306 determines that the at least one CO₂ parameter drops by the predetermined amount in the predetermined amount of time concurrently with the predetermined drop in delivered volume and/or the predetermined drop in exhaled volume, the method 300 performs alarm operation 308. If determination operation 306 determines that the at least one CO₂ parameter does not drop by the predetermined amount in the predetermined amount of time or concurrently the delivered volume and/or exhaled volume does not drop by their predetermined amounts, the method 300 returns to the monitoring operations 302, 304.

In one embodiment, method 300 receives the predetermined amount of VCO₂, ETCO₂, exhaled volume, and/or delivered volume from operator input. In another embodiment, the predetermined amounts are input by the operator. In an additional embodiment, the predetermined amounts are selected by the operator. In an alternative embodiment, the predetermined amounts are preconfigured and determined by the ventilator.

Additionally, method 300 performs alarm operation 308. Alarm operation 308 executes an occlusion alarm. The occlusion alarm signifies that the breathing circuit or patient interface is occluded. The occlusion alarm may be any suitable notification for gaining the attention of the medical caregiver, the ventilator operation, and/or the patient. In one embodiment, the occlusion alarm is a visual, audio, and/or vibrational notification.

In one embodiment, method 300 performs a display operation. Display operation displays the occlusion alarm on a ventilator display. In another embodiment, display operation of method 300 displays the occlusion alarm on a capnometer display.

In one embodiment, method 300 is performed by the medical ventilator-capnometer system illustrated in FIG. 1 and described above.

In an alternative embodiment, a computer-readable medium having computer-executable instructions for performing methods for managing the ventilation of a patient being ventilated by a medical ventilator-capnometer system are disclosed. These methods include repeatedly performing the steps illustrated in FIG. 3 and as described in the description of FIG. 3 above.

In another embodiment, a medical ventilator-capnometer system is disclosed. The medical ventilator-capnometer system includes: means for monitoring at least one CO₂ parameter, the at least one CO₂ parameter comprises ETCO₂ and VCO₂; means for monitoring at least one of exhaled volume and delivered volume; means for determining that the at least one CO₂ parameter drops by a predetermined amount in a predetermined amount of time concurrently with a drop in the at least one of the exhaled volume by a predetermined amount and the delivered volume by a predetermined amount; and means for executing an occlusion alarm. In one embodiment, the means for the medical ventilator-capnometer system are illustrated in FIG. 1 and described in the above description of FIG. 1. However, the means described above for FIG. 1 and illustrated in FIG. 1 are exemplary only and are not meant to be limiting.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for managing ventilation of a patient being ventilated by a medical ventilator, the method comprising:
monitoring at least one CO₂ parameter;
monitoring breathing circuit pressure;
monitoring exhaled flow and calculating exhaled volume therefrom;
determining that the at least one CO₂ parameter is less than a predetermined CO₂ threshold amount, the exhaled pressure is less than a predetermined threshold pressure, and the exhaled volume is less than a predetermined threshold volume; and
executing a disconnection alarm.

2. The method of claim 1, wherein the at least one CO₂ parameter comprises at least one of ETCO₂ and VCO₂.

3. The method of claim 1, further comprising:
displaying the disconnection alarm on at least one of a ventilator display or a capnometer display.

4. The method of claim 1, wherein at least one of the predetermined CO₂ threshold amount, the predetermined threshold pressure, and the predetermined threshold volume is received from operator input.

5. A method for managing ventilation of a patient being ventilated by a medical ventilator, the method comprising:
monitoring at least one CO₂ parameter of gas in the patient circuit;
monitoring at least one of exhaled volume and delivered volume;
determining that the at least one CO₂ parameter drops by a predetermined amount in a predetermined amount of time concurrently with a drop in the at least one of the exhaled volume by a predetermined amount and the delivered volume by a predetermined amount; and
executing an occlusion alarm.

6. The method of claim 5, wherein the at least one CO₂ parameter comprises at least one of ETCO₂ and VCO₂.

7. The method of claim 5, further comprising:
displaying the occlusion alarm on at least one a ventilator display or a capnometer display

8. The method of claim 5, wherein at least one of the predetermined drop amount of the at least one CO2 parameter, the predetermined amount of time, and the predetermined drop amount of the exhaled volume and the delivered volume is received from operator input.

9. A medical ventilator-capnometer system, comprising:
a pneumatic gas delivery system, the pneumatic gas delivery system adapted to control a flow of gas from a gas supply to a patient via a ventilator breathing circuit;
a flow sensor;
a pressure sensor;
a capnometer, the capnometer monitors an amount of carbon dioxide in respiration gas from the patient in the ventilator breathing circuit in order to monitor VCO₂ and ETCO₂;
a breathing circuit module, the breathing circuit module determines that concurrently at least one of the VCO₂ and the ETCO₂ are below a predetermined amount, pressure is below a predetermined amount, and an exhaled volume is below a predetermined amount in the ventilator breathing circuit based on flow sensor readings, pressure sensor readings, and capnometer readings before executing a disconnection alarm; and
a processor in communication with the pneumatic gas delivery system, the flow sensor, the pressure sensor, the capnometer, and the breathing circuit module.

10. The medical ventilator-capnometer system of claim 9, further comprising:
at least one of a ventilator display and a capnometer display.

11. The medical ventilator-capnometer system of claim 9, further comprising:
at least one of a visual disconnection alarm, an audio disconnection alarm, and a vibrational disconnection alarm.

12. The medical ventilator-capnometer system of claim 9, further comprising:
an operator interface, the operator interface allows an operator to select and input at least one of the predetermined amount of VCO₂, the predetermined amount of ETCO₂, the predetermined amount of pressure, and the predetermined amount of exhaled volume.

13. A medical ventilator-capnometer system, comprising:
a pneumatic gas delivery system, the pneumatic gas delivery system adapted to control a flow of gas from a gas supply to a patient via a ventilator breathing circuit;
a flow sensor;
a capnometer, the capnometer monitors an amount of carbon dioxide in respiration gas from the patient in the ventilator breathing circuit in order to monitor VCO₂ and ETCO₂;
a breathing circuit module, the breathing circuit module determines that at least one of the VCO₂ and the ETCO₂ drops by a predetermined amount within a predetermined amount of time, concurrently as at least one of delivered volume and exhaled volume drop by a predetermined amount in the ventilator breathing circuit based on flow sensor readings and capnometer readings before executing an occlusion alarm; and
a processor in communication with the pneumatic gas delivery system, the flow sensor, the capnometer, and the breathing circuit module.

14. The medical ventilator-capnometer system of claim 13, further comprising:
at least one of a ventilator display and a capnometer display.

15. The medical ventilator-capnometer system of claim 13, further comprising:
at least one of a visual occlusion alarm, an audio occlusion alarm, and a vibrational occlusion alarm.

16. The medical ventilator-capnometer system of claim 13, further comprising:
an operator interface, the operator interface allows an operator to select and input at least one of the predetermined drop amount of the VCO₂, the predetermined drop amount of the ETCO₂, the predetermined amount of time, the predetermined drop amount of the exhaled volume, and the predetermined drop amount of the delivered volume.

17. A computer-readable medium having computer-executable instructions for performing a method for managing ventilation of a patient being ventilated by a medical ventilator-capnometer system, the method comprising:
repeatedly monitoring at least one CO₂ parameter, the at least one CO₂ parameter comprises ETCO₂ and VCO₂;
repeatedly monitoring breathing circuit pressure;
repeatedly monitoring exhaled volume;
repeatedly determining that the at least one CO₂ parameter is less than a predetermined threshold amount, the exhaled pressure is less than a predetermined pressure threshold, and the exhaled volume is less than a predetermined volume threshold; and
repeatedly executing a disconnection alarm.

18. A medical ventilator-capnometer system, comprising:
means for monitoring at least one CO₂ parameter, the at least one CO₂ parameter comprises ETCO₂ and VCO₂;
means for monitoring at least one of exhaled volume and delivered volume;
means for determining that the at least one CO₂ parameter drops by a predetermined amount in a predetermined amount of time concurrently with a drop in the at least one of the exhaled volume by a predetermined amount and the delivered volume by a predetermined amount; and
means for executing an occlusion alarm.
